(19) United States
(12) Patent Application Publication
(10) Pub. No.: US 2021/0378586 A1
(43) Pub. Date: Dec. 9, 2021

(54) SKIN TANNING MEASURING DEVICE

(71) Applicant: Oren Aharon, Haifa (IL)

(72) Inventor: Oren Aharon, Haifa (IL)

(21) Appl. No.: 16/896,246

(22) Filed: Jun. 9, 2020

(51) Int. Cl. A61B 5/00; G01J 3/28; G01J 3/46

(52) U.S. Cl. CPC A61B 5/443; G01J 3/28; A61B 2562/0238; A61B 5/0082; A61B 5/486; G01J 3/46

(57) ABSTRACT

A Skin Tanning Measuring Device is provided to determine tanning level and skin penetration to various wavelengths, comprising of a device capable to inject light into the skin and monitor the light level emerging out from the skin tissue at a different location. By measuring the amount of emerging light, an individual tanning level could be deducted based on at least one additional measurement performed at an untanned location. The measurements will be used to compare between those levels and the saturated tanning levels, and will issue out a warning when the tanning is too high and additional exposure may be hazardous. Moreover, the offered system will measure skin penetration to UV hazardous radiation with and without sunscreen protection. The device will also provide information regarding melanin absorption as a function of UV exposure, and monitor those values over time and issue out a warning to users.

### BACKGROUND OF THE INVENTION

### Field of the Invention

The present disclosure is related to a system and method for characterizing the skin tanning and the absorption levels of its melanin to deduct the hazard level of a specific user to additional sun exposure. The tanning level is related to the amount of UV radiation exposure and it varies between individuals. The comparison between the tanning saturation level and the current level of tanning is a very good indication for UV exposure the person has been exposed to so far. The device will measure the exposure level, inform the user about it and warn in cases that the exposure level is too high. One should keep in mind that UV radiation is essential for a person's well-being for vitamin D synthesis. Tanning acts as a naturally produced sunscreen, and it is beneficiary for heliotherapy and phototherapy. It is an immunomodulator, improves cardiovascular health, and UV exposure may improve mood through the release of endorphins. Therefore, it is beneficial to have UV exposure, however, over-exposure has many negative effects, including skin cancer, skin-aging, eye damage, etc. It is thus important to conduct an assessment and follow-up of UV radiation exposure before clinical effects are detected, and to warn if clinical effects are observed. A smart AI software will be capable to evaluate sunscreen impact and protection for a specific user and will offer preventive measures. This proposed system can be used to match the level of sunscreen protection and overall behaviour to an individual user by monitoring the personal skin types and level of UV exposure.

### Description of the Related Art

Ultraviolet radiation has a great positive and negative impact on human beings' health including tanning, carcinogenesis, immunomodulation, and synthesis of vitamin D, among others. However, its impact is strongly dependent on the genetics of the person itself, his skin type and his environment. Thus, it is essential to adjust the needed UV exposure to an individual in order to avoid over or under-protection; over-protection will cause low production of vitamin D, affect the immune system, the mood which can have adverse health impact, and under protection may cause skin cancer and accelerate skin aging. UV exposure causes tanning of the skin, increased wrinkling and loss of elasticity. By following these indicators and factors for a specific user we can estimate the UV radiation dosage and the level of protection that is needed.

Today's technology does not enable measurement of UV impact tailored to a specific person, but rather will measure radiation levels created in surrounding environment and give a general warning—the best warning is adapted to inputs by user based on their skin colour. It is the purpose of this invention to offer a UV radiation exposure measurement device that is tailored to a specific user and will be capable of measuring UV exposure, impact of sunscreen protection, and identify skin areas with over exposure of UV radiation.

### SUMMARY OF THE INVENTION

In a preferred embodiment, a ring-like device is provided that injects light from its perimeter at different wavelengths into the skin. The wavelengths' radiation is back-emitted from central portion of said ring and collected by a detector system. Comparison between the emitted light level in an examined area and a referenced untanned area will yield the skin penetration or the amount of absorption of melanin, to evaluate the amount of melanin at the tanned skin area. The said detector system could be a camera for in-depth evaluation of skin quality and colour. Another preferred embodiment is to have a different ring-like device with a combination of light injecting devices and detectors mounted together on ring's periphery. This device includes upper illumination with UV sources, will measure skin penetration and melanin absorption, using said mounted detectors in the ring's periphery. The skin penetration will be measured using scattering of the upper illumination within the skin, and detection of the traveling light by said detectors that are mounted in the ring periphery. It should be clear that the plurality of light-source wavelength and different detectors could be designed according to the user skin type and quality. The embodiment includes at least one of the following:
A) Calculation algorithm comparing light absorption of an untanned skin area to the examined area.
B) Long term algorithm comparing penetration of a tanned area over time.
C) UV exposure assessment at the time the tanned area was checked.
D) Skin penetration when illuminated with UVA or UVB.
E) Skin quality and evaluation of damages.
F) An algorithm to warn when skin-tanned features are close to UV saturation or over exposed to UV radiation.

### BRIEF DESCRIPTION OF DRAWINGS

FIG. 1 describes a preferred embodiment of proposed device.

FIG. 2 describes yet another preferred embodiment of proposed device.

### DETAILED DESCRIPTION OF DRAWINGS

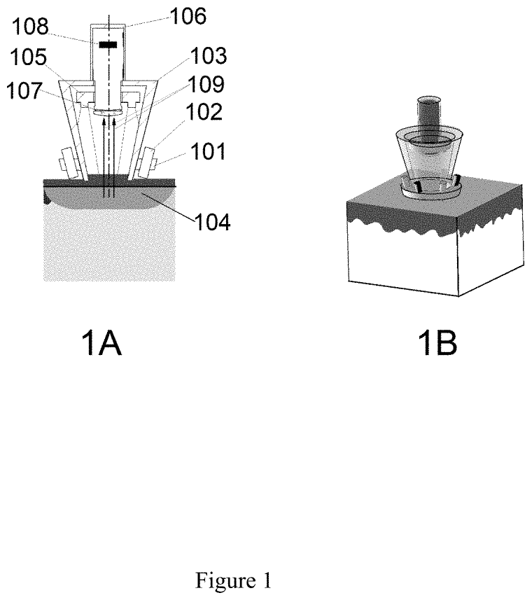

FIG. 1A is a preferred embodiment presented as a cross section of the 3-d picture denoted as 1B. This preferred embodiment is equipped with two illumination devices: 101 is based on a ring equipped with several light projectors denoted as 102, projecting multiple beams around an enclosed area where the tanned skin section is to be measured. The enclosure denoted as 103 is opaque. The illumination penetrates through the skin and creates a lobe denoted as 104 which illuminates the skin from underneath, and then penetrates through the center of enclosed area and travels along the direction denoted as 109, allowing calculation of total light penetrating through the skin to be computed. The light collection is performed by lens denoted 107 and focused on to a light detector denoted as 108. The system is also equipped with another illumination ring denoted as 105 which illuminates the skin from above to allow imaging with both types of illuminations. By choosing illumination wavelength, skin penetration is calculated as a function of wavelength. Calculating the level of melanin absorbance and skin tanning is performed by measuring two different skin areas—one tanned and the other with no tan.

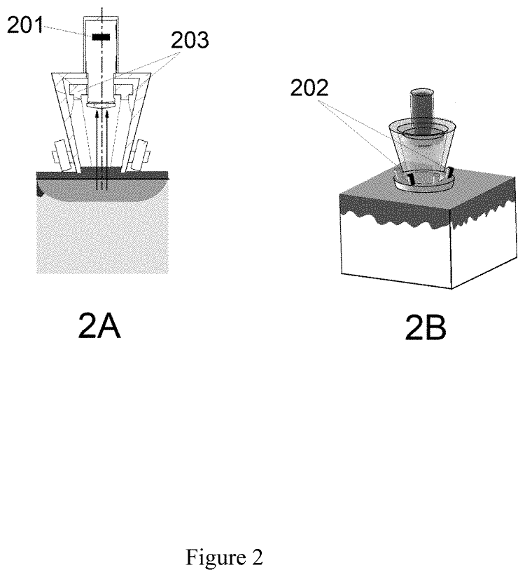

FIG. 2A is yet another embodiment wherein the detector 201 is an image detector, sensitive to the wavelength produced by said LED projectors. At the ring periphery, some detectors denoted as 202 were deployed to measure radiation produced by upper illumination denoted as 203 penetrating through the skin and reaching detector areas, thus measuring total skin penetration. Same said illumination is used to produce an image of enclosed skin area, the image is produced by imaging detector 201 to be further processed to create an image of skin and its damaged situation.

### General Description

Although the detailed description contains many specifics for the purpose of illustration, a person of ordinary skill in the art will appreciate that many variations and alterations to the design details are within the scope of the invention. Very narrow and specific examples are used to illustrate particular embodiments; however, the invention described in the claims is not intended to be limited to only these examples, but rather includes the full scope of the attached claims. Accordingly, the following preferred embodiments of the invention are set forth without any loss of generality to, and without imposing limitations upon the claimed invention.

The above detailed description of the preferred embodiments is accompanied by drawings that form a part hereof, and in which specific embodiments are shown in an illustrative way. It is understood that other embodiments may be utilized and structural changes may be made without departing from the scope of the present invention.

For ease of explanation the present invention is described as embodiments of an apparatus and method for illuminating under the skin area which is to be measured, and from back penetrating light to calculate the skin penetration. By measuring skin penetration in an untanned area and comparing it to the penetration of a tanned area, one can deduct the amount of tanning and its level from total tanning saturation. Since tanning is generated by UV radiation levels (UVA & UVB), then the total UV dosage can be formulated. In case dosage is close to saturation level, a warning of high UV exposure dosage will be issued. Moreover, total skin penetration can be deducted from outer illumination of the skin, and monitoring the penetrating light levels through the skin, and deducting what's the UV light penetration. This procedure could be used for skin with sunscreen or without, to figure out how effective the sunscreen protection is.

The device could be used as an over the counter tool or as a specialized diagnostic tool. The imaging device observing the skin will produce exact images of upper skin level and combined with information of penetration a full examination of skin health and UV dosage so far will be formalized.

A typical system operation is as follows:

### To Summarize

An apparatus comprising a diagnostic imaging device to be hand held, that will include an opaque tubular profile with an orifice adapted to be applied on a human skin portion. First light-emitting sources that are disposed around the orifice tube wall will illuminate towards the skin and around the periphery of the tubular profile, to emit radiation penetrating the skin and illuminating the enclosed skin portion by under the skin scattering. Second light emitting sources will illuminate the skin from above and inside the opaque tubular profile. A detection system will be used for recording the back-light illumination penetrating through the enclosed skin portion and calculate the melanin absorption, deducting the UV dosage the skin was exposed so far. Using past information of skin's maximum tanning saturation or melanin absorbance, we deduct the skin condition regarding to UV exposure. Information will be processed and displayed using current computer technologies or displayed and calculated on a personal mobile phone system. Another application of proposed apparatus will be to measure and suggest sunscreen minimal level to protect an individual based on his/her skin type and condition.

What is claimed is:

1. A device configured to measure skin tan and its melanin absorbance level comprising of:
a mechanical ring that includes a plurality of different wavelengths light sources, wherein the ring is configured to be applied to an enclosed skin section in its center;
a detection system disposed above the ring center and configured to measure the light penetration through said enclosed skin center; and
a computing device calculating the said penetrating light level and comparing results to a reference non-tanned skin measurement.

2. The apparatus of claim 1 further comprising an upper illumination source to illuminate said enclosed skin area from above, to measure skin reflection and light scattering within said enclosed skin tissue using imaging.

3. The apparatus of claim 1 further comprising an imaging device as a detection system from above.

4. The apparatus of claim 1 further comprising detector elements disposed on the ring perimeter to examine light travels within said enclosed skin tissue from said above illumination.

5. The apparatus of claim 1 comprising an additional computing device configured to capture the imaging data from the detection system, to measure skin tanning and UV exposure and compare it to past measurements.

6. The apparatus of claim 1 wherein said computing device is configured to save information, perform follow-up measurements and suggest UV protection level based on skin type and UV exposure.
